(19) United States
(12) Patent Application Publication
(10) Pub. No.: US 2014/0208476 A1
(43) Pub. Date: Jul. 31, 2014

(54) Welding helmet having a dual vision arrangement

(71) Applicants: Huang Chi Chen, (US); Ching Che Chen, (US); Ying Cheng Chen, (US); Sheng Yuan Chen, (US)

(72) Inventor: Huang Chi Chen, Changhua (TW)

(73) Assignees: Huang Chi Chen, Changhua (TW); Sheng Yuan Chen, Changhua (TW); Ying Cheng Chen, Changhua (TW); Ching Che Chen, Changhua (TW)

(21) Appl. No.: 13/753,488

(22) Filed: Jan. 29, 2013

(51) Int. Cl. A61F 9/06

(52) U.S. Cl. CPC A61F 9/06
USPC 2/8.7; 2/8.2

(57) ABSTRACT

A welding helmet is provided with a housing including a pivotal head band, a forward window, and a shield disposed on the window and including an opening being forwardly of the window; and a visor including a frame secured to the housing, an upper tinted lens disposed on the frame, a lower transparent lens disposed on the frame, the lower transparent lens being at an angle with respect to the upper tinted lens, and a pivotal panel disposed between two sides of the shield to separate the opening into two parts. A wearer may view the tinted lens under welding conditions or view the transparent lens after welding.

### BACKGROUND OF THE INVENTION

1. Field of the Invention

The invention relates to welding helmets and more particularly to a welding helmet having a dual vision arrangement so that a wearer may view a tinted lens under welding conditions or view a transparent lens after welding.

2. Description of Related Art

There is a conventional welding helmet comprising a mask including first and second viewing windows, the first viewing window having a tinted lens adapted to view under welding conditions and the second viewing window having a relatively transparent lens adapted to view work to be welded prior to striking an arc. The tinted lens is mounted forward of the face of a wearer. The relatively transparent lens extends at an angle rearward towards the wearer away from the plane of the tinted lens and at an acute angle with respect to the normal line of vision of the wearer through the tinted lens. The angle is sufficient to substantially reduce the amount of light entering into the mask through the second viewing window when the wearer is viewing a light source, located at arm's length along the normal line of vision through the first viewing window.

Notwithstanding the prior art, the invention is neither taught nor rendered obvious thereby.

### SUMMARY OF THE INVENTION

It is therefore one object of the invention to provide a welding helmet comprising a housing comprising a pivotal head band, a forward window, and a shield disposed on the window and including an opening being forwardly of the window; and a visor comprising a frame secured to the housing, an upper tinted lens disposed on the frame, a lower transparent lens disposed on the frame, the lower transparent lens being at an angle with respect to the upper tinted lens, and a pivotal panel disposed between two sides of the shield to separate the opening into two parts.

The above and other objects, features and advantages of the invention will become apparent from the following detailed description taken with the accompanying drawings.

### BRIEF DESCRIPTION OF THE DRAWINGS

FIG. 1 is a perspective view of a welding helmet according to a first preferred embodiment of the invention;

FIG. 2 is an exploded view of the welding helmet;

FIG. 3 is a longitudinal sectional view of the welding helmet;

FIG. 4 is a view similar to FIG. 3 showing the welding helmet worn on a wearer and the wearer viewing through the tinted lens under welding conditions;

FIG. 5 is a view similar to FIG. 4 showing the wearer viewing a workpiece through the transparent lens after welding;

FIG. 6 is a perspective view of a welding helmet according to a second preferred embodiment of the invention; and

FIG. 7 is an exploded view of the welding helmet of FIG. 6.

### DETAILED DESCRIPTION OF THE INVENTION

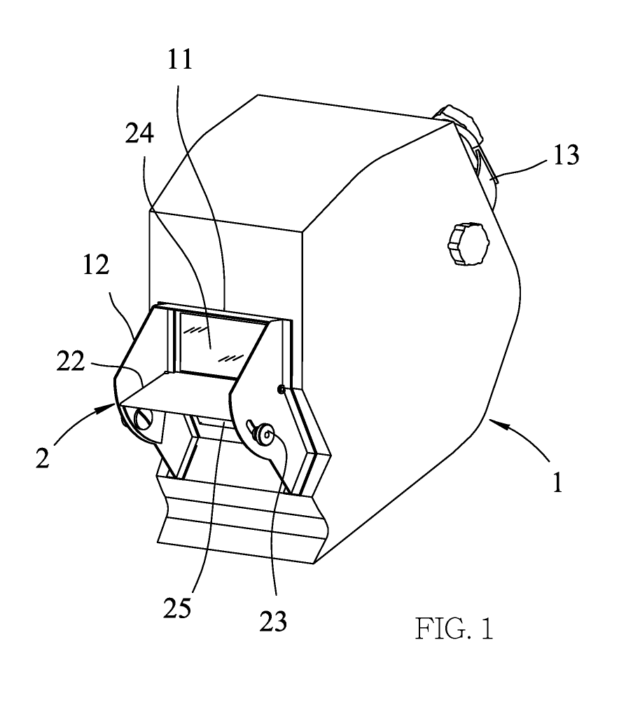

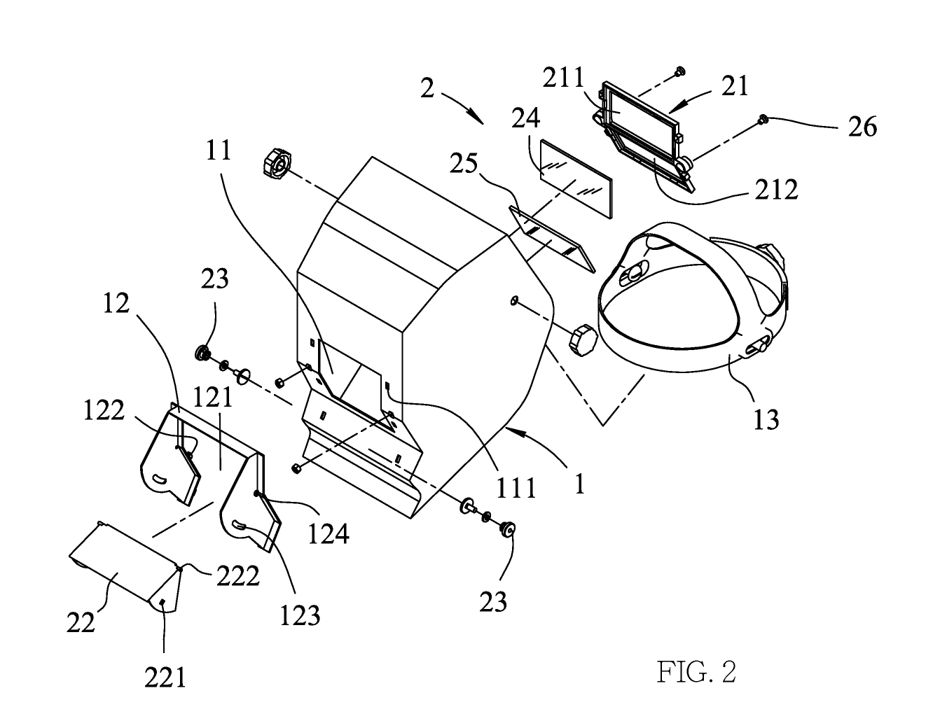

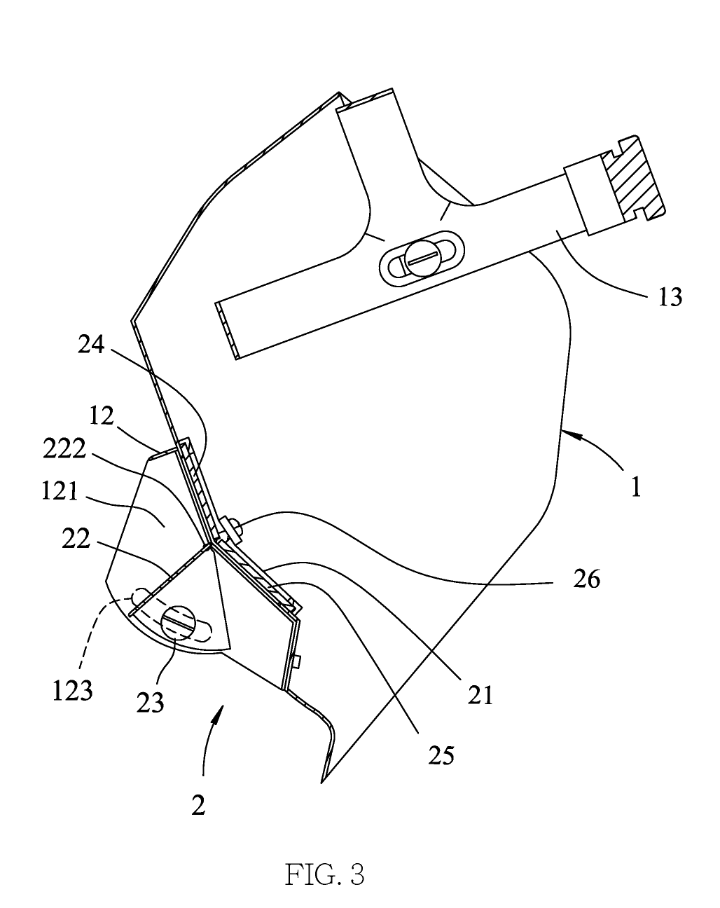

Referring to FIGS. 1 to 5, a welding helmet in accordance with a first preferred embodiment of the invention comprises the following components as discussed in detail below.

A housing 1 has an open bottom and is adapted to completely cover a wearer's face. The housing 1 comprises a supporting head band 13 pivotably secured to an upper portion. The housing 1 further comprises a forward shield 12 including an opening 121, two groups of three pins 122 on rear surfaces of both sides respectively, two holes 124 on front surfaces of both sides respectively, and two elongated slots 123 on both sides of the shield 12 respectively; a window 11 disposed on a front surface of the housing 1, and two groups of three holes 111 on both sides of the window 11 respectively. The pins 122 are inserted into the holes 111 to secure the shield 12 to the housing 1.

A visor 2 comprises a frame 21 for defining an upper opening 211 and a lower opening 212 which is at an angle with respect to the upper opening 211, a tinted lens 24 disposed on the upper opening 211, a transparent lens (or a filter lens) 25 disposed on the lower opening 212; a forward bent panel 22 disposed in the shield 12 and including two holes 221 on both sides respectively, and two pivotal pins 222 on both ends of a rear edge respectively; two sliding adjustment members 23 inserted through the slots 123 and the holes 221 to slidably secure the bent panel 22 to the shield 12; and a fastener 26 for securing the frame 21 to the housing 1. The pivotal pins 222 are inserted into the holes 124 to pivotably secure the bent panel 22 to the shield 12.

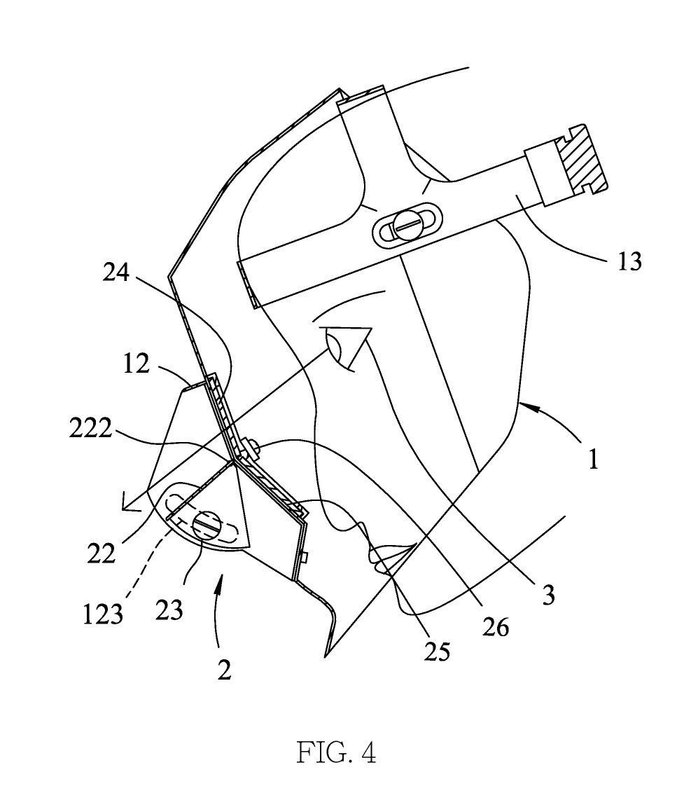

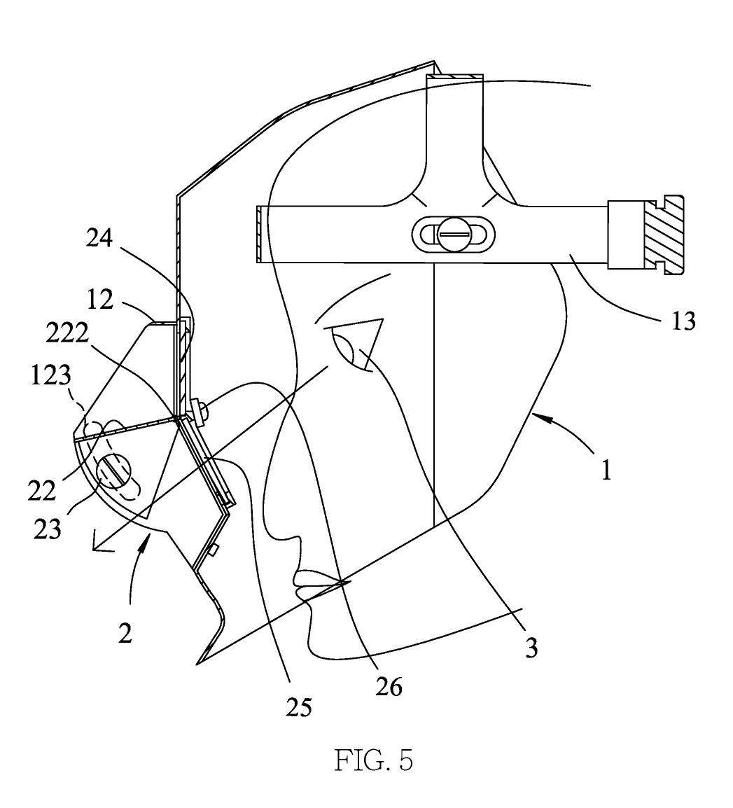

Referring to FIG. 4 specifically, the eyes 3 of a wearer may view through the tinted lens 24 to see a workpiece to be welded (see field of view indicated by arrow) under welding conditions by downward tilting his/her head. Referring to FIG. 5 specifically, after welding the wearer may upward tilt the head from the position of FIG. 4 to an about horizontal position of FIG. 5. Next, the wearer may view through the transparent lens 25 to see the workpiece (see field of view as indicated by arrow) for inspection.

It is envisaged by the invention that the wearer can slide the sliding adjustment members 23 along the slots 123 downward to increase the space above the bent panel 22 prior to welding or slide the sliding adjustment members 23 upward to increase the space below the bent panel 22 after welding. Above operation can increase the field of view to clearly see the workpiece with a minimum tilt of the head.

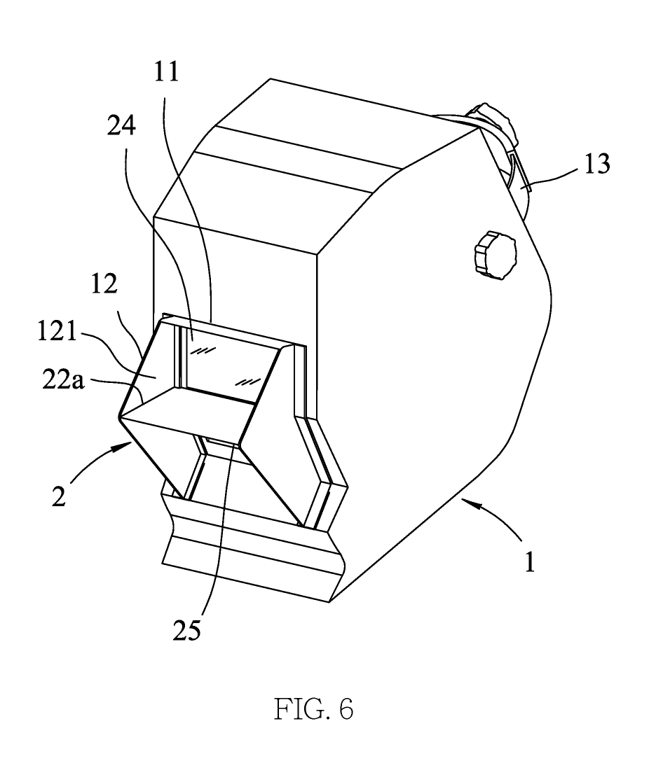

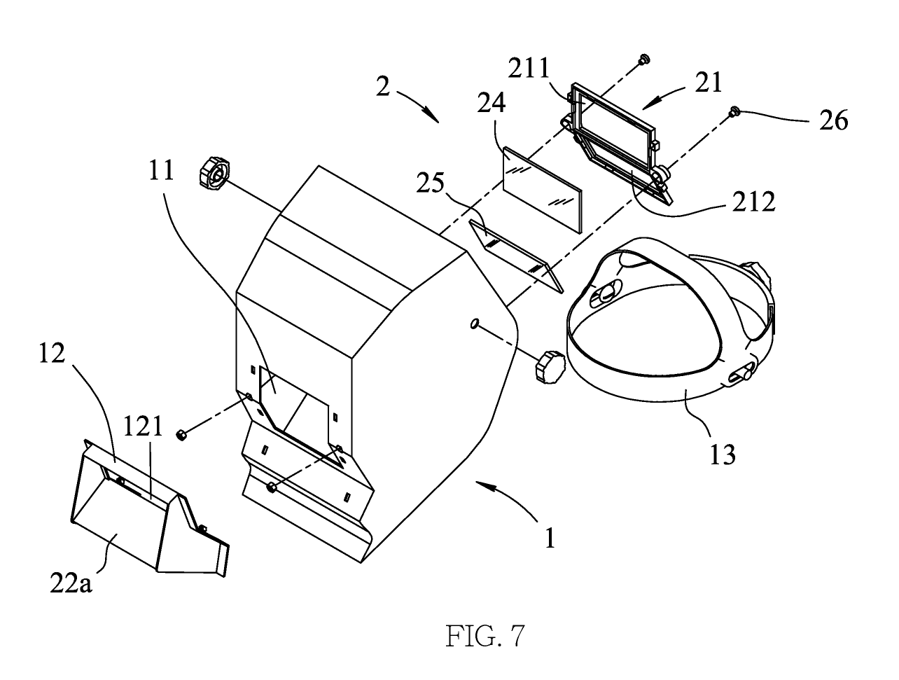

Referring to FIGS. 6 and 7, a welding helmet in accordance with a second preferred embodiment of the invention is shown. The characteristics of the second preferred embodiment are substantially the same as that of the first preferred embodiment except the following: An inclined panel 22a is permanently secured to the shield 12 with the sliding adjustment member being removed.

While the invention has been described in terms of preferred embodiments, those skilled in the art will recognize that the invention can be practiced with modifications within the spirit and scope of the appended claims.

What is claimed is:

1. A welding helmet comprising:
a housing comprising a pivotal head band, a forward window, and a shield disposed on the window and including an opening being forwardly of the window; and
a visor comprising a frame secured to the housing, an upper tinted lens disposed on the frame, a lower predetermined lens disposed on the frame, the lower predetermined lens being at an angle with respect to the upper tinted lens, and a pivotal panel disposed between two sides of the shield to separate the opening into two parts.

2. The welding helmet of claim 1, wherein the lower predetermined lens is a transparent lens.

3. The welding helmet of claim 1, wherein the lower predetermined lens is a filter lens.

4. The welding helmet of claim 1, wherein the shield further comprises two holes on front surfaces of both sides respectively, and wherein the pivotal panel comprises two pivotal pins on both ends of a rear edge respectively, the pivotal pins being inserted into the holes to pivotably secure the pivotal panel to the shield.

5. A welding helmet comprising:
a housing comprising a pivotal head band, a forward window, and a shield disposed on the window and including an opening being forwardly of the window; and
a visor comprising a frame secured to the housing, an upper predetermined lens disposed on the frame, a lower tinted lens disposed on the frame, the upper predetermined lens being at an angle with respect to the lower tinted lens, and a pivotal panel disposed between two sides of the shield to separate the opening into two parts.

6. The welding helmet of claim 5, wherein the upper predetermined lens is a transparent lens.

7. The welding helmet of claim 5, wherein the upper predetermined lens is a filter lens.

8. The welding helmet of claim 5, wherein the shield further comprises two holes on front surfaces of both sides respectively, and wherein the pivotal panel comprises two pivotal pins on both ends of a rear edge respectively, the pivotal pins being inserted into the holes to pivotably secure the pivotal panel to the shield.

9. A welding helmet comprising:
a housing comprising a pivotal head band, a forward window, and a shield disposed on the window and including an opening being forwardly of the window; and
a visor comprising a frame secured to the housing, an upper predetermined lens disposed on the frame, a lower tinted lens disposed on the frame, the upper predetermined lens being at an angle with respect to the lower tinted lens, and a panel disposed between two sides of the shield to separate the opening into two parts.

10. The welding helmet of claim 9, wherein the upper predetermined lens is a transparent lens.

11. The welding helmet of claim 9, wherein the upper predetermined lens is a filter lens.
